(19) United States
(12) Patent Application Publication
(10) Pub. No.: US 2022/0088079 A1
(43) Pub. Date: Mar. 24, 2022

(54) METABOLITE ENCAPSULATING NANOPARTICLES TO ENHANCE CELLULAR CANCER IMMUNOTHERAPY

(71) Applicant: TECHNION RESEARCH & DEVELOPMENT FOUNDATION LIMITED, Haifa (IL)

(72) Inventors: Tomer SHLOMI, Kfar-Hes (IL); Abraham RUTENBERG, Haifa (IL)

(21) Appl. No.: 17/530,193

(22) Filed: Nov. 18, 2021

Related U.S. Application Data
(63) Continuation of application No. PCT/IL2020/050543, filed on May 19, 2020.
(60) Provisional application No. 62/889,107, filed on Aug. 20, 2019.
(60) Provisional application No. 62/850,036, filed on May 20, 2019.

(51) Int. Cl. A61K 35/17; A61K 9/51; A61P 35/00; C12N 5/0783; A61K 45/06

(52) U.S. Cl. CPC A61K 35/17; A61K 9/5115; A61P 35/00; B82Y 5/00; C12N 5/0646; A61K 45/06; C12N 5/0636

(57) ABSTRACT

Provided herein are nanoparticles encapsulating a required substance for enriching immune cells with the required substance, compositions comprising the same and methods for their preparation. Further provided are methods of using the nanoparticles for enriching the immune cells with the required substance as well as methods of treating cancer using the nanoparticles or compositions comprising the same.

### CROSS REFERENCE TO RELATED APPLICATIONS

This application is a Continuation of PCT Patent Application No. PCT/IL2020/050543 having International filing date of May 19, 2020, entitled “METABOLITE ENCAPSULATING NANOPARTICLES TO ENHANCE CELLULAR CANCER IMMUNOTHERAPY”, which claims the benefit of priority under 35 U.S.C. § 119(e) of U.S. Provisional Application No. 62/889,107, filed on Aug. 20, 2019, entitled “METABOLITE ENCAPSULATING NANOPARTICLES TO ENHANCE T CELL CANCER IMMUNOTHERAPY and U.S. Provisional Application No. 62/850,036, filed on May 20, 2019, entitled “NOVEL CANCER IMMUNOTHERAPY APPROACH: NANOPARTICLE BASED FEEDING OF T CELLS WITH METABOLIC NUTRIENTS TO OVERCOME METABOLIC IMMUNE SUPPRESSION”. The contents of the above applications are all incorporated by reference as if fully set forth herein in their entirety

### BACKGROUND OF THE INVENTION

Cancer immunotherapy has become well established in recent years as one of the most promising approaches in cancer treatment and potential cure. Patients with late-stage metastatic cancers that were considered until few years ago incurable show durable responses when treated with commercial immune checkpoint inhibitors (unleashing T cells from signaling suppression) or with the commercial cellular therapies of Chimeric Antigen Receptor (CAR) T cells. However, for most patients and in most cancer types the clinical benefit of immunotherapy is temporarily. Finally, relapse occurs or disease progresses. Cured patients with complete response comprise only a small minority of patients especially in solid tumors. In some cancer types, even objective response rates to immunotherapies remains low including for common breast and prostate cancers. Furthermore, CAR T therapy was clinically proven effective and is currently FDA approved only for specific hematological cancers and was not yet approved for solid tumors. Other immunotherapy strategies including adoptive tumor-infiltrating lymphocyte (TIL) therapy and adoptive CAR natural killer cells (NK) therapy have yet to gain FDA approval.

Major progress has been made in recent years in the identification of metabolic rewiring of cancer cells. Numerous metabolic pathways have been found to be changed in their level of activity following tumorigenic transformation. Alterations in tumor cell metabolism greatly affects metabolite availability in the tumor microenvironment. For example, induced aerobic glycolysis in tumors (‘Warburg effect’) was shown to deplete glucose from the tumor microenvironment. Depletion of various amino acids including arginine, glutamine, cysteine, serine, tryptophan and others, has been recently highlighted. Recent papers suggest that depletion of these key metabolic nutrients results in metabolic suppression, i.e., leads to T cell starvation, hampers T cell effector functions, and prevents these cells from undergoing effective activation, differentiation and proliferation. This leads to T cell suppression, anergy and apoptosis, and consequent tumor escape. Similar findings of metabolic suppression were recently described also for NK cells. While cancer immunotherapy R&D has focused mostly on identifying inhibitory signaling pathways and developing products that can overcome such inhibitions (immune checkpoint inhibitors), little attention has been given to metabolic suppression of immune cells and how it might potentially be relieved to improve immune cell function in immunotherapy.

Overcoming metabolic suppression in the tumor microenvironment is technically challenging in that both immune cells (e.g. T cells or NK cells) and cancer cells within a tumor compete for the same metabolic nutrients. A notable example is arginine, which is of particular importance for T cell and NK function. Arginine is degraded in the tumor microenvironment in various cancers via arginase (secreted by neutrophils and immature myeloid cells), which leads to low arginine concentrations, resulting in T cell and/or NK suppression and arrested proliferation. For overcoming arginine scarcity, arginase inhibitors were shown to increase arginine levels in the tumor microenvironment and to partially restore T cell functions. In fact, arginase inhibitors developed by Calithera Biosciences are in phase 1/2 clinical trials for solid tumors. On the other hand, the capacity to synthesize arginine is damaged in many solid tumors, due to silencing of ASS1 (arginosuccinate synthase 1), thus making them dependent on exogenous arginine supply. Hence, arginine depletion is considered a viable therapeutic approach (e.g., Polaris Pharma developing a PEGylated phase 3 arginine deiminase and Aeglea Biotherapeutics is developing a phase 1 arginase). This paradox of whether arginine levels should be elevated or dropped emphasizes the problem of having to supply metabolic nutrients just to immune cells such as T or NK cells without feeding cancer. The problem illustrated by this paradox is general not only for arginine but for any metabolite upon which the tumor competes with immune cells, such as, T cells and NK cells. Current adoptive cell transfer therapies of T cells, NK cells, CART cells, CAR NK cells and TILs offer no effective solution for this essential nutrient competition and depletion at the tumor microenvironment. Moreover, there is currently no solution to systemically deliver or target metabolic nutrients to a patients' immune cells even as not as a part of an adaptive cell transfer. Metabolic immune suppression is one of the major obstacles accounting for the limited success of cancer immunotherapy.

### SUMMARY OF THE INVENTION

The invention provides a nanotechnology-based solution to overcome immune cell metabolic suppression, which is of particular importance in cancer. According to some embodiments, disclosed herein are controlled release nanoparticles optionally from silica shell encapsulating a required substance, (such as, a required metabolite, sugar, amino acid, nucleoside, nucleotide, ribonucleoside, ribonucleotide, nutrient or combination thereof), essential for immune cell activation. The required substance is loaded using the nanoparticles to immune cells which may be modified and/or unmodified immune cells. The nanoparticles may gradually release their metabolic cargo in the cells, thus supporting the immune cell metabolic requirements over time, reducing immune cell starvation at the tumor microenvironment and enabling better effector functions and anti-tumor response. The nanoparticles can be used to metabolically enhance immune cells which are used in adoptive immune cell transfer therapies like CAR T, CAR NK or TIL therapies, or be systemically targeted to internal (natural occurring i.e. unmodified) immune cells, resulting in superior cancer immunotherapy. In some embodiments, methods of the invention for treating cancer using the nanoparticles, can be combined with other cancer treatments.

In some embodiments of the invention, there is provided a method of enriching immune cells with a required substance, the method comprising the step of contacting the immune cells with a nanoparticle comprising the required substance, thereby enriching the immune cells with the required substance.

In some embodiments of the invention, the nanoparticle comprises an encapsulating shell or a nanosphere.

In some embodiments of the invention, the required substance may be or comprises at least about 5% w/w of the weight of the nanoparticle.

In some embodiments of the invention, the required substance is at a weight of at least about 5, 10, 15, 20, 25, 30% or more w/w of the nanoparticle. In some embodiments, the weight ratio (w/w) between the required substance and the nanoparticle refers to a nano particle which does not include additional targeting agents, as detailed below.

In some embodiments of the invention, the required substance comprises a required essential metabolite, sugar, amino acid, nucleoside, nucleotide, ribonucleoside, ribonucleotide, nutrient, or any combination thereof.

In some embodiments of the invention, the required substance comprises a required essential metabolite, sugar, amino acid, nutrient, or any combination thereof.

In some embodiments of the invention, the required substance comprises a sugar. In some embodiments, the sugar is glucose.

In some embodiments of the invention, the required substance comprises an amino acid. In some embodiments, the amino acid may include: arginine, glutamine, serine, tryptophan, alanine, methionine and/or glycine. Each possibility is a separate embodiment.

In some embodiments of the invention, the enrichment of the immune cells with the required substance enhances one or more of: activity, viability, potency, life span or function of the immune cells. Each possibility is a separate embodiment.

In some embodiments of the invention, the nanoparticle is contacted with the immune cells during an ex-vivo stage or is targeted to internal immune cells by systemic administration.

In some embodiments of the invention, the immune cells are selected from the group consisting of: T cells, NK cells, CAR T cells, CAR NK cells, TIL cells, or any combination thereof. Each possibility is a separate embodiment.

In some embodiments of the invention, the nanoparticle is capable of gradually releasing the required substance into the immune cells.

In some embodiments of the invention, the nanoparticle comprises silica.

In some embodiments of the invention, the silica is non-porous, porous, semi-porous, macro-porous, meso-porous, or combinations thereof.

In some embodiments of the invention, silica is amorphous, crystalline or semi-crystalline.

In some embodiments of the invention, the silica is polymerized using a sol-gel polymerization process.

In some embodiments of the invention, the nanoparticle is coated with or attached to an immune cell targeting agent.

In some embodiments of the invention, the immune cell targeting agent is selected from the group consisting of an antibody, peptide, aptamer, heptamer, oligomer, targeting vector, and combinations thereof.

In some embodiments of the invention, the antibody is selected from the group consisting of an anti-CD3 antibody, anti-CD2 antibody, anti-CD4 antibody, anti-CD8 antibody, anti-PD1 antibody, anti-CTLA4 antibody, anti-KIR antibody, anti-CD16 antibody, anti-CD94 antibody, anti-CD161 antibody, anti-CD56 antibody, anti-NTBA antibody, recombinant human NTBA, and any combination thereof.

In some embodiments of the invention, there is provided a nanoparticle for enriching immune cells with a required substance, the nanoparticle comprising a silica shell.

In some embodiments of the invention, the nanoparticle shell is capable of encapsulating the required substance.

In some embodiments of the invention, the required substance comprises a required metabolite, sugar, amino acid, nucleoside, nucleotide, ribonucleoside, ribonucleotide, nutrient, or the combination thereof.

In some embodiments of the invention, the required substance comprises a required essential metabolite, sugar, amino acid, nutrient, or any combination thereof.

In some embodiments of the invention, the required substance comprises glucose.

In some embodiments of the invention, the required substance comprises arginine, glutamine, serine, tryptophan, alanine, methionine and/or glycine.

In some embodiments of the invention, the silica is selected from non-porous, porous, semi-porous, macro-porous, meso-porous, or combinations thereof.

In some embodiments of the invention, the silica is amorphous, crystalline or semi-crystalline.

In some embodiments of the invention, the silica is polymerized using a sol-gel polymerization process.

In some embodiments of the invention, the nanoparticle is coated by or attached to an immune cell targeting agent, wherein the immune cell targeting agent is an antibody, peptide, aptamer, heptamer, oligomer, targeting vector, nanobody, or any combination thereof.

In some embodiments of the invention, the antibody is an anti-CD3 antibody, anti-CD2 antibody, anti-CD4 antibody, anti-CD8 antibody, anti-PD1 antibody, anti-CTLA4 antibody, anti-KIR antibody, anti-CD16 antibody, anti-CD94 antibody, anti-CD161 antibody, anti-CD56 antibody, anti-NTBA antibody, recombinant human NTBA, or any combination thereof.

In some embodiments of the invention, the nanoparticle is capable of releasing the required substance within the immune cells in a controlled manner with a time frame of 0 minutes-24 hours or 2-10 days.

In some embodiments of the invention, the nanoparticle is capable of delivering the required substance to the immune cells.

In some embodiments of the invention, there is provided a composition comprising a plurality of the nanoparticles according to the embodiments of the invention.

In some embodiments of the invention, there is provided a nanoparticle for enriching immune cells with sugar and/or an amino acid, the nanoparticle comprising a shell capable of encapsulating the sugar and/or the amino acid, wherein the nanoparticle is coated by or attached to one or more immune cell targeting agents, wherein the one or more targeting agent is selected from an antibody, peptide, aptamer, heptamer, oligomer, targeting vector or nanobody.

In some embodiments of the invention, the antibody is an anti-CD3 antibody, anti-CD2 antibody, anti-CD4 antibody, anti-CD8 antibody, anti-PD1 antibody, anti-CTLA4 antibody, anti-KIR antibody, anti-CD16 antibody, anti-CD94 antibody, anti-CD161 antibody, anti-CD56 antibody, anti-NTBA antibody, recombinant human NTBA, or any combination thereof.

In some embodiments of the invention, the nanoparticle is capable of releasing the sugar and/or the amino acid in a controlled manner with a time frame of 0 minutes-24 hours or 2-10 days.

In some embodiments of the invention, the nanoparticle shell comprises silica.

In some embodiments of the invention, the silica is selected from non-porous, porous, semi-porous, macro-porous, meso-porous, or combinations thereof.

In some embodiments of the invention, the silica is amorphous, crystalline or semi-crystalline.

In some embodiments of the invention, the silica is polymerized using a sol-gel polymerization process.

In some embodiments of the invention, there is provided a composition comprising a plurality of the nanoparticles as described above for enriching immune cells with sugar and/or an amino acid, the nanoparticles comprising a shell capable of encapsulating the sugar and/or the amino acid, wherein the nanoparticles is coated by or attached to one or more immune cell targeting agents, wherein the one or more targeting agent is selected from an antibody, peptide, aptamer, heptamer, oligomer, targeting vector or nanobody.

In some embodiments of the invention, there is provided a method of treating cancer in a subject in need, the method comprising the steps of contacting ex vivo one or more types of immune cells with the nanoparticle of the invention, the nanoparticle comprising a required substance; and administering to the subject in need the immune cells comprising said nanoparticle.

In some embodiments of the invention, there is provided a method of treating cancer in a subject in need, the method comprising the step of administering systematically or locally to the subject in need the nanoparticle or the composition comprising the same.

In some embodiments of the invention, there is provided a method of treating cancer in a subject in need, the method comprising the steps of contacting ex vivo one or more types of immune cells with the nanoparticle of the invention, the nanoparticle comprising a sugar and/or amino acid; and administering to the subject in need the immune cells comprising the nanoparticles.

In some embodiments of the invention, there is provided a method of treating cancer in a subject in need, the method comprising the step of administering systematically or locally to the subject in need the nanoparticle according to the invention, or the composition comprising the same.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the drawings:

FIGS. 1A and B illustrate the effect of metabolite encapsulating nanoparticles when the encapsulated metabolite in an exemplary embodiment of the invention, is arginine. Arginine depletion in the tumor microenvironment by secreted human arginase 1 suppresses T cell functions (FIG. 1A). Arginase secreted by MDSCs and neutrophils in the tumor microenvironment or induced arginine consumption by MDSCs and neutrophils deplete arginine resulting in T cell metabolic suppression. Suppression reversal by arginine nanoparticles fed to T cells is shown in FIG. 1B.

FIGS. 2 A and B: FIG. 2A shows current procedure of adoptive CAR T therapy. FIG. 2B shows metabolite encapsulating nanoparticles feeding of essential metabolites to CAR T cells in CAR T therapy. Nanoparticles are fed to CAR T cells during the ex-vivo phase of therapy, then metabolically enriched CAR T cells are infused to the patient.

FIGS. 3 A, B, C, D, E, F, G, H, I, J, K and L: FIG. 3A is an exemplary framework for core-shell nanoparticles synthetic approach in which a silica shell is grown on a nano-meter sized core of the desired metabolite for encapsulation. Arginine an exemplary embodiment: Commercially available arginine (FIG. 3A) is grinded to nano-scale using high energy ball mill grinding (FIG. 3B). A silica shell is then polymerized using a sol-gel process on the arginine nano-powder resulting in encapsulating arginine in its core, forming core-shell nanoparticles (FIGS. 3C and 3D). Nanoparticles are derivatized using an organically modified alkoxysilane or silane to insert organic functionally (e.g. amine groups, carboxyl groups, hydroxyl groups, epoxy groups, cyano groups, thiol groups and the like) to which an antibody can be conjugated to facilitate T cell uptake (FIG. 3E). In the shown example APTES ((3-Aminopropyl)triethoxysilane) is used as derivatizing reagent to insert amine functionality. Finally, an antibody is conjugated to the nanoparticles to enable uptake by target cells (FIG. 3F). In the scheme, an anti-CD3 antibody is conjugated to enable T cell and CAR T cell uptake. FIGS. 3 G, H, I and J show examples of different thicknesses of the encapsulating silica shell and exemplary dimensions of the arginine core: (FIG. 3G) 15-18 nm shell; (FIG. 3H) 30-38 nm shell on a 162×142 nm core; (FIGS. 31 and 3J) 47-50 nm shell encapsulating 121×153 and 88×76 nm cores; (FIGS. 3K and 3L) HRSTEM EDX (High Resolution Scanning Transmission Electron Microscopy Energy-Dispersive X-ray Spectroscopy) confirming nano-metric arginine core encapsulated in silica shell shows silicon atoms of the silica shell encompassing carbon and nitrogen atoms of the arginine core. Images acquired using Zeiss Ultra-Plus HRSEM and FEI Tecnai G2 T20 TEM, The Electron Microscopy Center (MIKA), Technion. HRSTEM EDX acquired using FEI Titan Cubed Themis G² 60-300, The Electron Microscopy Center (MIKA), Technion.

FIGS. 4 A, B, C, D, E, F and G: FIG. 4A is a framework for nanoparticle synthetic approach of metabolite encapsulation in Stober-process silica nanoparticles, core-shell nanoparticles of a silica shell grown on a silica core, emulsion polymerization silica nanoparticles and hollow silica nanospheres. General scheme for silica nanoparticle synthesis, arginine (or other metabolite) encapsulation and antibody conjugation to enable T or NK cell uptake. FIGS. 4B, 4C, 4D, 4E, 4F and 4G show metabolite encapsulating silica nanoparticles synthesized by the inventors: FIG. 4B shows Stober-process 350 nm silica nanoparticles; FIG. 4C shows Stober-process 600 nm silica nanoparticles; FIG. 4D shows silica shell grown on silica core 50 nm nanoparticles; (FIG. 3E) emulsion polymerization 50 nm nanoparticles; FIG. 4F shows 200 nm silica nanospheres obtained by chemical etching of polystyrene core with toluene; FIG. 4G shows 200 nm silica nanospheres obtained by thermal etching of polystyrene core. Images acquired using Zeiss Ultra-Plus HRSEM and FEI Tecnai G2 T20 TEM, The Electron Microscopy Center (MIKA), Technion.

FIG. 5 shows the controlled release kinetics for arginine and glucose from silica nanoparticles as measured by LC-MS (SeQuant ZIC-pHILIC column; Thermo Q-Exactive mass spectrometer with an electrospray ionization source).

FIGS. 6 A, B and C show FITC-containing silica 600 nm nanoparticle entry to Jurkat T cells depends on anti-CD3 coating. (FIG. 6A) Flow cytometry: Data acquired using BD LSR-II analyzer at the LS&E infrastructure center and analyzed using FlowJo software. FIGS. 6B and 6C are photographs from confocal microscopy: Nanoparticles in green (FITC), Nucleus in blue (DAPI), actin fibers in red (Phalloidin). Acquired using Confocal Zeiss LSM 710, LS&E Infrastructure unit, Technion.

FIG. 7 shows arginine depletion suppressing effect on human CD8+ T cell activation. Absence of arginine during activation increases T cell apoptosis, arrests proliferation and reduces the expression of the activation markers 4-1BB and CD25. Data acquired using BD LSR-II analyzer at the LS&E infrastructure center and analyzed using FlowJo software.

FIG. 8 shows that arginine loaded nanoparticles partially restores T cell activation in an arginine depleted medium. Control nanoparticles containing no arginine and two types of arginine encapsulating nanoparticles (designated type 1 and type 2) were fed to CD8 T cells before transfer to an arginine depleted medium followed by an anti-CD3/anti-CD28 stimulation. 4-1BB activation marker expression measured by flow cytometry suggests superior T cell activation when T cells are fed with arginine containing nanoparticles vs. controls.

### DESCRIPTION OF THE DETAILED EMBODIMENTS

Tumors compete with immune cells, such as, T cells and NK cells entering the tumor microenvironment and deprive them from essential metabolic nutrients including glucose, glutamine, arginine and other amino acids, sugars, nucleotides and other nutrients. The immune cells starvation occurring at the tumor microenvironment hampers their functions including activation, differentiation and killing abilities. This results in immune cells (for example without limitation, T cell and/or NK cell) suppression and anergy, leading to tumor escape. The immune cells may be modified or unmodified, i.e. may be cells that were genetically modified ex-vivo or internal cells of the immune system.

The terms “immune cells”, “immune cell”, “modified and/or unmodified immune cell/s”, or “cells that are used in immunotherapy” interchangeably define herein modified and/or unmodified. The term unmodified immune cells refers here to naturally occurring immune cells such as naturally occurring T cells and natural killer (NK) cells, as well as tumor-infiltrating lymphocyte (TILs), B cells, monocytes, macrophages, dendritic cells, neutrophils, eosinophils, basophils, any type of leukocytes or a combination thereof. The term modified immune cells refers to genetically engineered cells such as CAR T cells or CAR NK cells. In an embodiment of the invention, the immune cells function is suppressed by nutrient scarcity.

The terms “enriched immune cells”, “enriched immune cell”, enriched modified or unmodified immune cell/s, or “enriched cells that are used in immunotherapy” interchangeably define modified and/or unmodified immune cells, inserted with or attached to the nanoparticles, nanospheres, or the encapsulating shell comprising the required substance as defined herein.

In some embodiments of the invention, the terms “treatment,” “treat,” and “treating” refer to reversing, alleviating, ameliorating, or inhibiting the progress of the disease, or one or more symptoms thereof or restoring or partially restoring the activation, function or life span of the immune cells.

In some embodiments of the invention, the cancer is a solid tumor cancer.

In some embodiments of the invention, the required substance is at a weight of at least 5% w/w of the nanoparticle.

In some embodiments of the invention, the required substance is at a weight of at least 5, 10, 15, 20, 25, 30% or more w/w of the nanoparticle.

In some embodiments, the weight ratio between the encapsulated substance and the nanoparticle refers to a nanoparticle which does not include or attached to immune cell targeting agent.

According to some embodiments, there is provided a nanotechnology-based solution to overcome immune cell metabolic suppression at the tumor microenvironment. Controlled release nanoparticles or nanospheres, optionally from silica, encapsulating a required metabolite, sugar, amino acid, nucleoside, nucleotide, ribonucleoside, ribonucleotide, nutrient or combination thereof, for immune cell activation are loaded to modified or unmodified immune cells. The nanoparticles can be loaded to the modified or unmodified immune cells during the ex-vivo stage of adoptive immune cells transfer (for example, for CAR T, CAR NK or TIL therapy), or targeted and delivered systemically to modified or unmodified immune cells. Upon uptake, the nanoparticles create a depot of nutrients inside the immune cells. The nanoparticles can gradually release the encapsulated metabolites inside the immune cells, providing the cells with internal food supply, thus removing/reducing/reversing the metabolic suppression imposed by the tumor and enabling superior effector functions. This approach of nanoparticle based metabolic feeding of modified or unmodified immune cells, such as, T cells and/or NK cells can act as a standalone therapy or be combined with immune checkpoint inhibitors, CAR T therapy, CAR NK therapy, TIL therapy, cancer vaccines, other types of cancer immunotherapy approaches or non-immunotherapy approaches, most notably chemotherapy, biological therapies like tyrosine kinase inhibitors, anti-angiogenic therapy, hormonal therapy, radiotherapy, surgery and the like.

In some embodiments of the invention, there is further provided a nanotechnology-based solution to overcome cell metabolic suppression at the tumor microenvironment. Controlled release nanoparticles or nanospheres having a silica shell encapsulating a required substance which may be an essential required metabolite, sugar, amino acid, nucleoside, nucleotide, ribonucleoside, ribonucleotide, nutrient or combination thereof, for cell activation, are loaded to the cells that are used for cancer cell therapy. The cells may be any cells, such as, kidney cells, liver cells, stem cells and the like.

In some embodiments of the invention, there is further provided a nanotechnology-based solution to overcome a cell metabolic suppression. Controlled release nanoparticles or nanospheres optionally from silica, encapsulating a substance, which is an essential required metabolite, sugar, amino acid, nucleoside, nucleotide, ribonucleoside, ribonucleotide, nutrient or combination thereof for cell activation are loaded to the cells that are used for cell therapy. The cells may be any cells, such as, kidney cells, liver cells, stem cells and the like.

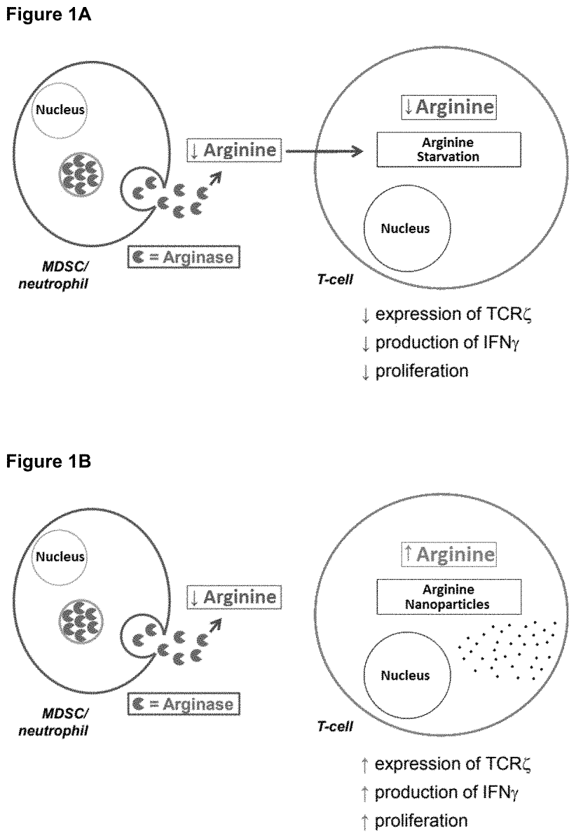

In some embodiments, the invention comprises a nanotechnology-based approach to overcome metabolic immune cell suppression in cellular cancer immunotherapy: specifically delivering essential metabolic nutrients to modified and/or unmodified immune cells via nanoparticle encapsulation. This approach maintains an internal reservoir of essential nutrients inside the T cell and/or NK cell, supplying its needs and supporting its metabolism when the T cell and/or NK cell reaches the nutrient-limited tumor microenvironment. This approach also facilitates the metabolic suppression of T cells and/or NK cells already residing within the tumor microenvironment when the nanoparticles are systemically delivered and targeted to those cells. The nutrient support supplied by the metabolite encapsulating nanoparticles relives the metabolic suppression of T cells and/or NK cells, enhance their activation and effector function to eliminate the tumor. By a way of example, FIG. 1 illustrates the effect of metabolite encapsulating nanoparticles when the encapsulated metabolite is the amino acid arginine.

According to some embodiments, the essential metabolic nutrients (required substance) for relieving metabolic immune cell (such as, T cell, NK cell, CAR T, CAR NK and/or TILs) suppression, include such substances as, but not limited to: glucose, fructose, galactose, glycerol, glutamine, glutamate, arginine, citrulline, serine, cysteine, tryptophan, alanine, histidine, lysine, aspartic acid, glutamic acid, threonine, asparagine, selenocysteine, glycine, proline, valine, leucine, isoleucine, methionine, phenylalanine, tyrosine, NAD, NADH (nicotinamide adenine dinucleotide), FAD, FADH2 (flavin adenine dinucleotide), glycolysis intermediates: glucose-6-phosphate, fructose-6-phosphate, fructose 1,6-biphosphate, dihydroxyacetone phosphate, glycerol 3-phosphate, 1,3-bisphosphoglycerate, 3-phosphoglycerate, 2-phosphoglycerate, phosphoenolpyruvate, pyruvate, citric acid cycle (Krebs cycle) intermediates: acetyl-CoA, citrate, cis-aconitate, isocitrate, alpha-ketoglutarate, succinyl-CoA, succinate, fumarate, malate, oxaloacetate, one-carbon metabolic pathway intermediates, folate cycle intermediates, methionine cycle intermediates, trans-sulfuration pathway intermediates, urea cycle intermediates, nucleotide synthesis pathway intermediates, nucleotide or nucleoside triphosphate, diphosphate or monophosphate, ribonucleotide or ribonucleoside triphosphate, diphosphate or monophosphate, ATP (adenosine triphosphate), GTP, CTP, UTP, TTP, dGTP, dCTP, dUTP or dTTP and the like, or any combination thereof. Each possibility is a separate embodiment. In some embodiments, the required substances disclosed herein may be generally defined in the application as “required metabolite, sugar, amino acid, nucleoside, nucleotide, ribonucleoside, ribonucleotide or a nutrient”. Encapsulating nanoparticles may contain in some embodiments of the invention a single metabolite/single type of metabolite or combination of metabolites/types of metabolites.

In an embodiment of the invention, there is provided a nanoparticle or nanosphere coated by or attached to immune cells, such as without limitation, T cell or NK cell targeting agent, wherein the nanoparticle or the nanosphere or encapsulating shell are loaded with a required substance, which is an essential metabolite, sugar, amino acid, nucleoside, nucleotide, ribonucleoside, ribonucleotide or nutrient or a combination thereof.

The targeting agent may be an antibody, peptide, aptamer, heptamer, oligomer, targeting vector or nanobody.

In some embodiments of the invention, the antibody is a T cell specific antibody, such as, for example without limitation, anti-CD3 antibody, anti-CD4 antibody, anti-CD8 antibody, anti-PD1 antibody, anti-CTLA4 antibody, or NK cell specific antibody for example, anti-KIR antibody, anti-CD16 antibody, anti-CD94 antibody, anti-CD161 antibody, anti-CD56 antibody and the like or a combination of thereof for dual targeting or an antibody or a protein targeting a common target for both T cells and NK cells for dual targeting such as, for example without limitation, recombinant human NTBA or anti-NTBA antibody.

In some embodiments of the invention, the nanoparticle, nanosphere or encapsulating shell is made of silica or organically modified silica. In some embodiments of the invention, polystyrene is used as a scaffold in a hard-templating approach to polymerize the silica nanosphere around it and then removed by chemical or thermal etching to create the void volume (core) to enable metabolite loading. In some embodiments of the invention, the nanoparticle, nanosphere or encapsulating shell are made of silica shell grown on the required metabolite, sugar, amino acid, nucleoside, nucleotide, ribonucleoside, ribonucleotide or the nutrient nano-powder. In some embodiments of the invention, the nanoparticle or nanosphere releases the metabolite, amino acid, nutrient or the combination thereof in a controlled manner with a time frame of 0 minutes-24 hours and/or at least 1, 2, 5, 7, 10, 12, 15, 27, 20 days or more. The release may initiate once the nanoparticle, nanosphere or encapsulating shell is in contact with a solution including water or cell media and the like.

In some embodiments of the invention, the nanoparticle or nanosphere or encapsulating shell releases the metabolite, sugar, amino acid, nucleoside, nucleotide, ribonucleoside, ribonucleotide, nutrient or the combination thereof in a controlled manner with a time frame of 0-60 minutes. In some embodiments, the release is in a time frame of at least 1, 2, 3, 4, 5, 6, 7, 8, 9, 10, 11, 12, 13, 14, 15, 16, 17, 18, 19, 20, 21, 22, 23 or 24 hours. In some embodiments of the invention, the nanoparticle or nanosphere or encapsulating shell releases the metabolite, sugar, amino acid, nucleoside, nucleotide, ribonucleoside, ribonucleotide, nutrient or the combination thereof in a controlled manner with a time frame of more than 1, 2, 3, 4, 5, 6, 7, 8, 9, 10, 11, 12, 13, 14, 15, 16, 17, 18, 19, or 20 days or more.

In some embodiments of the invention, the nanoparticle or the nanosphere is a non-porous silica nanoparticle, a porous or semi-porous silica nanoparticle, core-shell nanoparticle, silica hollow sphere or coated silica shell.

In some embodiments of the invention, there is provided a modified and/or not modified immune cell, such as, T Cell, NK cell CAR T cell, CAR NK cell or TILs containing inside, or externally attached, to the nanoparticle, nanosphere or encapsulating shell of the invention.

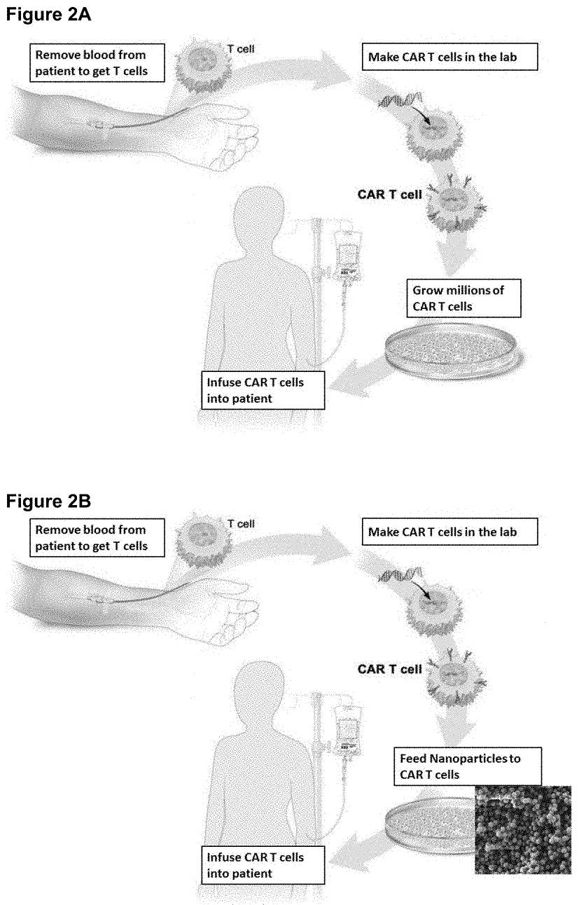

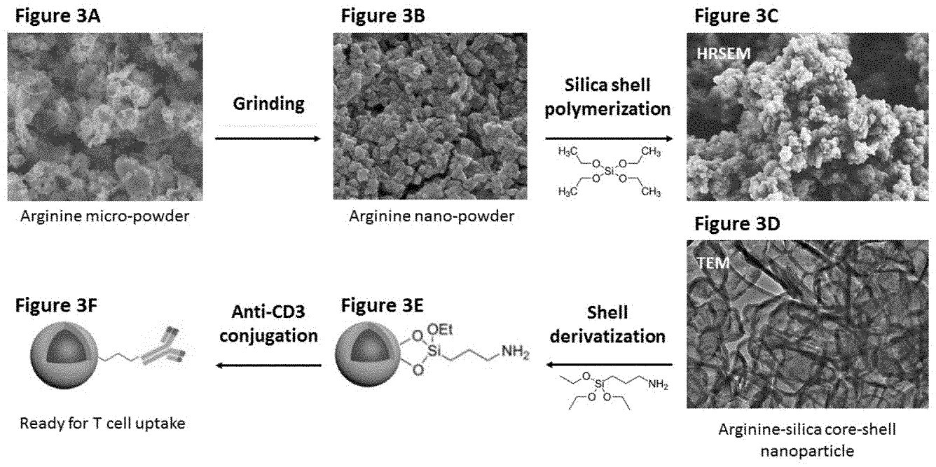

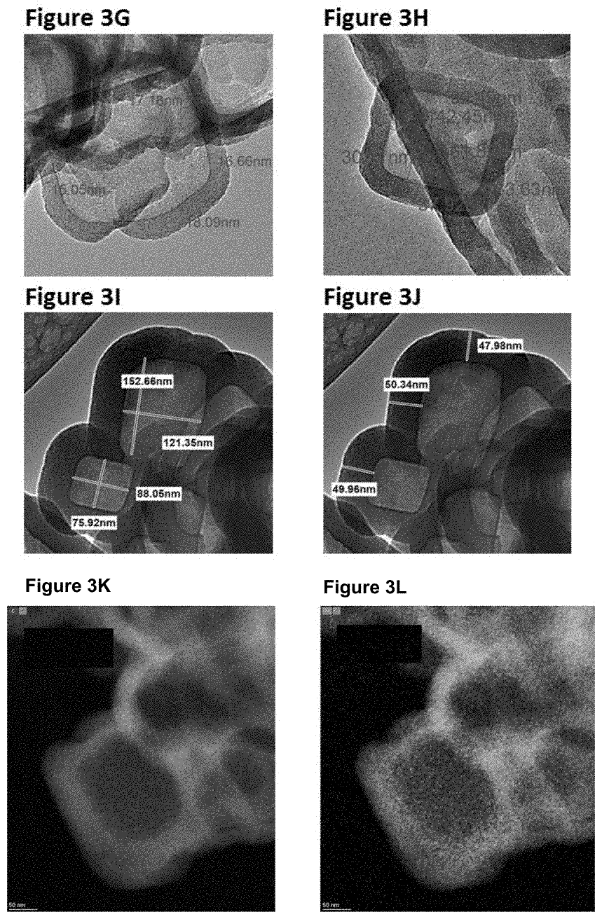

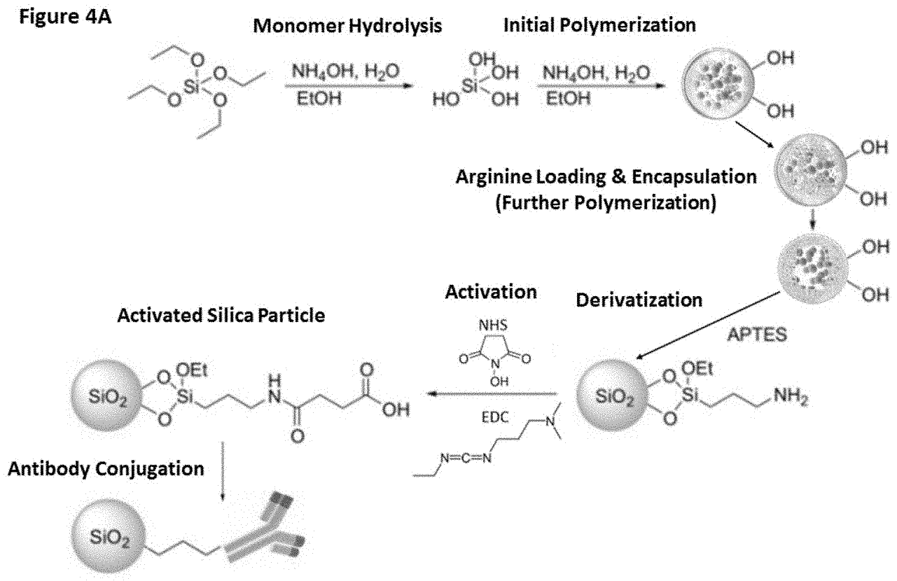

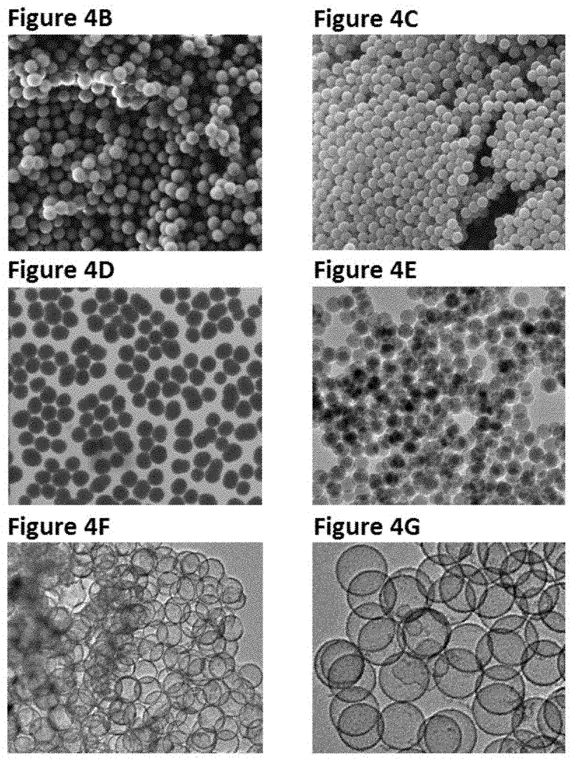

The nanoparticles, nanospheres or encapsulating shell can be delivered systemically (e.g. intravenous administration), while specificity and T cell and/or NK cell targeting may be achieved by coating the nanoparticles with a targeting agent as described above, such as for example, T cell specific antibody (e.g. anti-CD3 antibody, anti-CD4 antibody, anti-CD8 antibody, anti-PD1 antibody, anti-CTLA4 antibody, etc.) or NK cell specific antibody (e.g. anti-KIR antibody, anti-CD16 antibody, anti-CD94 antibody, anti-CD161 antibody, anti-CD56 antibody, etc.) or a combination of thereof for dual targeting or an antibody or a protein targeting a common target for both T cells and NK cells for dual targeting (e.g. recombinant human NTBA or anti-NTBA antibody). The targeting agent can target the nanoparticles or nanospheres or encapsulating shell to all T cell and/or NK cell population (e.g. using anti-CD3 antibody for all T cell population) or to specific T cell and/or NK cell sub-populations (e.g. CD4 T cells using anti-CD4 antibody; CD8 T cells using anti-CD8 antibody; to activated or exhausted T cells using anti-PD1 antibody or anti-CTLA4 antibody, etc.). For the purpose of systemic delivery, the nanoparticles may be optionally PEGylated. Alternatively, the nanoparticles can be directly fed to T cells, NK cells, CAR T cells, CAR NK cells or TILs during the ex-vivo phase of adoptive T cell, NK cell, CAR T, CAR NK or TIL therapy. In this case the nanoparticles are simply added to the growth medium several hours before patient reinfusion as illustrated in FIG. 2. Also in this embodiment, the nanoparticles nanospheres or encapsulating shell may be coated with a targeting agent as described above, such as for example, an antibody to facilitate T cell uptake (e.g. anti-CD3 antibody) or NK cell uptake (e.g. an antibody or lectin).

The nanoparticles, nanospheres or encapsulating shell may be made, in some embodiments of the invention, from silica (such as silicon oxide), amorphous or crystalline organically modified or not and further may be designed so as to provide the encapsulated nutrients in a controlled release manner over a typical time frame of hours, days or weeks. In some embodiments, the silica nanoparticles are synthesized using a sol-gel polymerization process starting from precursors including TEOS (Tetraethyl orthosilicate), TMOS (Tetramethyl orthosilicate), sodium silicate, potassium silicate, alkoxysilanes, silanes, organically modified alkoxysilanes, organically modified silanes or any other silica precursor. The invention includes in some of its embodiments, nanoparticles with different porosity and nano-architectures including porous, semi-porous and non-porous silica nanoparticles, core-shell nanoparticles, silica hollow spheres, silica shell coating on nutrient nano-powders (e.g. growing silica shells on glucose, glutamine or arginine nano-powders) and mesoporous silica nanoparticles. The invention includes nanoparticles, nanospheres or encapsulating shell synthesized using the Stober methodology, emulsion polymerization, microemulsion polymerization, and silica shell grown on nano-powders of the metabolic nutrients directly (coating the metabolic nutrients with a silica shell). The invention further includes silica nanoparticles synthesized using the sol-gel polymerization process. In some embodiments, during the synthetic procedure, the encapsulated nutrient can be added to the reaction before, during or after the formation of the silica nanoparticles or shell. The silica can be derivatized using an organically modified alkoxysilane or organically modified silane to control hydrophilicity/hydrophobicity (effecting the encapsulated metabolite release rate), insert PEGylation or insert organic functionality (e.g. amine groups, carboxyl groups, hydroxyl groups, epoxy groups, cyano groups, thiol groups and the like) for the conjugation of antibodies or other targeting moieties to target the nanoparticles, nanospheres or encapsulating shell to the modified and/or unmodified immune cells.

In some embodiments of the invention, the nanoparticles may be made from PLGA, PGA, PLA, PLC, Liposomes, ethyl cellulose, casein, alginate, hydrogel, albumin, chitosan, emulsion, microemulsion, micelle, solid lipid, dendrimers, polylysine, poly(amidoamine), metallic nanoparticles, nanocrystals, and any combination thereof.

In some embodiments of the invention, there is provided a method of enriching modified and/or unmodified immune cells with a required metabolite, sugar, amino acid, nucleoside, nucleotide, ribonucleoside, ribonucleotide or nutrient or a combination thereof or a method of delivering into modified and/or unmodified immune cells a required metabolite, sugar, amino acid, nucleoside, nucleotide, ribonucleoside, ribonucleotide or nutrient or a combination thereof comprising the steps of: contacting the modified and/or unmodified immune cells with a nanoparticle or nanosphere encapsulating with, or attached to, the required metabolite, sugar, amino acid, nucleoside, nucleotide, ribonucleoside, ribonucleotide or nutrient or the combination thereof thereby enriching modified and/or unmodified immune cells with the required metabolite, sugar, amino acid, nucleoside, nucleotide, ribonucleoside, ribonucleotide or nutrient or a combination thereof or delivering into modified and/or unmodified immune cells the required metabolite, sugar, amino acid, nucleoside, nucleotide, ribonucleoside, ribonucleotide or nutrient or a combination thereof.

The nanoparticle or the nanosphere or encapsulating shell (those terms are used herein interchangeably) is contacted with the modified and/or unmodified immune cells either during the ex-vivo stage of CAR T, CAR NK, TIL or any other adoptive T cell, NK cell or TILs transfer therapy or by systemic administration.

In some embodiments of the invention, the nanoparticle, nanosphere or encapsulating shell gradually release the encapsulated required metabolite, sugar, amino acid, nucleoside, nucleotide, ribonucleoside, ribonucleotide or a nutrient.

In some embodiments of the invention, a depot of the required metabolite, sugar, amino acid, nucleoside, nucleotide, ribonucleoside, ribonucleotide or a nutrient is created inside the modified and/or unmodified immune cells.

In some embodiments of the invention, the nanoparticle or nanosphere or encapsulating shell are coated or attached to an immune cell (such as, T cells and/or NK cell) targeting agent as described above.

The invention includes the use of the proposed treatment as a standalone monotherapy or in combination with other cancer immunotherapies, most notably immune checkpoint inhibitors, adoptive T cell therapies, adoptive NK cell therapies, adoptive CAR T therapies, adoptive CAR NK therapies and adoptive TIL therapies, cancer vaccines, conjugated antibodies, bi-specific T cell engagers, bi-specific NK cell engagers, oncolytic viruses, ‘eat me’ signals, ‘find me’ signals or other types of cancer immunotherapy approaches or non-immunotherapy approaches, including chemotherapy, biological therapies like tyrosine kinase inhibitors, anti-angiogenic therapy, hormonal therapy, radiotherapy, and surgery.

In some embodiments of the invention, there is provided a method of manufacturing the nanoparticle or nanosphere of the invention, comprising the steps of:

mixing NH₄OH and an alcohol for alkaline catalysis or mixing an acid (e.g. HCl, HNO₃, H₂SO₄) and an alcohol for acid catalysis;
optionally adding water;
adding tetraethyl orthosilicate (TEOS) or tetramethyl orthosilicate (TMOS) or sodium silicate or potassium silicate or alkoxysilane or silane or organically modified alkoxysilane or organically modified silane;
suspending the obtained nanoparticle or nanosphere in a glycerol or alcohol solution and a required metabolite, amino acid, or nutrient or the combination thereof;
heating; and
lyophilizing.

In some embodiments of the invention, the alcohol is methanol, ethanol, 1-propanol, 2-propanol, butanol (linear or branched), pentanol (linear or branched), hexanol (linear or branched), a longer chain alcohol or a combination thereof.

In some embodiments of the invention, the heating is at a temperature at the range of 40° C.-240° C.

In some embodiments of the invention, the heating is at a temperature at the range of 50° C.-140° C.

In some embodiments of the invention, the heating is at a temperature at the range of 70° C.-90° C.

In some embodiments of the invention, the heating is at a temperature at the range of 75° C.-85° C.

In some embodiments of the invention, the heating is at about 80° C.

In some embodiments of the invention, there is provided a method of manufacturing the silica shell encapsulating metabolite nano-particles of the invention, comprising the steps of:

grinding desired metabolite to the nano-scale using an alcohol for wet grinding or grinding desired metabolite to the nano-scale using dry grinding and suspending the metabolite nano-powder in an alcohol;
optionally adding a base (e.g. NH₄OH) for alkaline catalysis or adding an acid (e.g. HCl, HNO₃, H₂SO₄) for acid catalysis;
optionally adding water;
adding tetraethyl orthosilicate (TEOS) or tetramethyl orthosilicate (TMOS) or sodium silicate or potassium silicate or alkoxysilane or silane or organically modified alkoxysilane or organically modified silane;
optionally heating;
separating the nanoparticles;
and lyophilizing.

In some embodiments of the invention, the alcohol is methanol, ethanol, 1-propanol, 2-propanol, butanol (linear or branched), pentanol (linear or branched), hexanol (linear or branched), a longer chain alcohol or a combination thereof.

In some embodiments of the invention, the separation is by centrifugation.

In some embodiments of the invention, there is provided a method for manufacturing a nanoparticle or nanosphere which is hollow sphere comprising the steps of:

mixing polyvinylpyrrolidone, styrene and water;
heating;
adding potassium persulfate or AMA (2,2′-Azobis(2-methylpropionamidine) dihydrochloride) as initiator;
cooling;
adding ammonium hydroxide (for alkaline catalysis) or an acid (for acid catalysis), alcohol and
TEOS or TMOS or sodium silicate or potassium silicate or alkoxysilane or silane or organically modified alkoxysilane or organically modified silane;
etching of the polystyrene core by calcination (above 350° C.) or by adding a solvent to dissolve the polystyrene core (e.g. toluene);
washing with an alcohol;
subjecting the formed hollow sphere to a metabolite, sugar, amino acid, nucleoside, nucleotide, ribonucleoside, ribonucleotide, nutrient or combination thereof to a loading solution (such as in water or any other solution in which the metabolite, sugar, amino acid, nucleoside, nucleotide, ribonucleoside, ribonucleotide, nutrient or combination thereof are easily dissolved);
heating;
drying; and optionally,
lyophilizing.

In some embodiments of the invention, if subjecting the formed hollow sphere to more than one metabolite, sugar, amino acid, nucleoside, nucleotide, ribonucleoside, ribonucleotide, nutrient or combination thereof, the process is conducted for each metabolite, sugar, amino acid, nucleoside, nucleotide, ribonucleoside, ribonucleotide or nutrient separately. In some embodiments of the invention, if subjecting the formed hollow sphere to more than one metabolite, sugar, amino acid, nucleoside, nucleotide, ribonucleoside, ribonucleotide, nutrient or combination thereof, the process is conducted for each metabolite, sugar, amino acid, nucleoside, nucleotide, ribonucleoside, ribonucleotide or nutrient jointly.

In some embodiments of the invention, the methods described herein, further comprise a step of derivatization of the resulted nanoparticle or nanosphere or encapsulating shell using an organically modified alkoxysilane or organically modified silane.

In some embodiments of the invention, the derivatization is done by suspending the nanoparticles or nanospheres or encapsulating shell in alcohol, for example as without being limited, ethanol or ethanol-water mixtures (e.g. 96% ethanol and 4% water). In some embodiments of the invention, APTES ((3-Aminopropyl)triethoxysilane or carboxyethylsilanetriol, is added. This can be done for 1, 2, 3, 4, 5, 6, 7, 8, 9, 10, 11 or 12 hours or more or 1, 2, 3, 4, 5, 6, 7, 8, 9, 10, 11, 12 days or more. The temperature is in some embodiments, between 15-240° C. In some embodiments, the temperature is between 50-200° C. In some embodiments, the temperature is between 60-100° C. In some embodiments, the nanoparticles or nanospheres or encapsulating shell are washed by ethanol or any other alcohol.

In some embodiments of the invention, there is provided a method of manufacturing nanoparticle, nanosphere or encapsulating shell, wherein a required metabolite, sugar, amino acid, nucleoside, nucleotide, ribonucleoside, ribonucleotide or a nutrient or combination thereof is encapsulated in a silica shell, comprising of steps:

mixing an alcohol with NH₄OH, NaOH, KOH or other strong or weak bases for alkaline catalysis or adding an acid HNO₃, H₂SO₄ or other strong or weak acids for acid catalysis;
optionally adding water;
adding tetraethyl orthosilicate (TEOS), tetramethyl orthosilicate (TMOS), an alkoxysilane, sodium silicate, potassium silicate, silane, organically modified alkoxysilane or organically modified silane;
optionally heating;
washing the nanoparticles with alcohol;
optionally separating the nanoparticles;
suspending the obtained nanoparticles a solution with a required metabolite, sugar, amino acid, nucleoside, nucleotide, ribonucleoside, ribonucleotide, nutrient or the combination thereof;
optionally heating;
optionally washing with water and/or alcohol; and
drying or lyophilizing.

In some embodiments of the invention water hydrolyzes the silica precursor followed by silica condensation. This includes water added as pure water to the reaction medium or as part of acid or base catalysis (e.g. diluted or concentrated acid or base that contains water).

In some embodiments of the invention, an organically modified alkoxysilane or an organically modified silane is derivatizing the nanoparticles, nanospheres or encapsulating shell to insert an organic functionality (e.g. amine groups, carboxyl groups, hydroxyl groups, epoxy groups, cyano groups, thiol groups and the like). In some embodiments of the invention, the nanoparticles, nanospheres or encapsulating shell are further PEGylated.

In some embodiments of the invention, the nanoparticles, nanospheres or encapsulated shell are manufactured by Stober process nanoparticles, emulsion polymerization nanoparticles, core-shell nanoparticles or hollow silica nanosphere.

In some embodiments of the invention, the nanoparticle is synthesized in a Stober-like process and the required metabolite, sugar, amino acid, nucleoside, nucleotide, ribonucleoside, ribonucleotide or nutrient or a combination thereof is encapsulated in the silica porous space.

In some embodiments of the invention, the nanoparticle is a core-shell structure made of a silica shell grown on a silica core, while the required metabolite, sugar, amino acid, nucleoside, nucleotide, ribonucleoside, ribonucleotide or nutrient or a combination thereof is encapsulated in the silica shell.

In some embodiments of the invention, the nanospheres encapsulate the metabolite, sugar, amino acid, nucleoside, nucleotide, ribonucleoside, ribonucleotide or nutrient in its hollow core while the shell is made of silica or organically modified silica.

In some embodiments of the invention, the nanoparticle is a core-shell structure made of a silica shell grown on a substrate core comprising the required metabolite, sugar, amino acid, nucleoside, nucleotide, ribonucleoside, ribonucleotide or nutrient or a combination thereof.

In some embodiments of the invention, a silica shell is polymerized on nano-powder of the desired metabolite, sugar, amino acid, nucleoside, nucleotide, ribonucleoside, ribonucleotide or nutrient to be encapsulated resulting in a core-shell architecture nanoparticles, in which the core is the desired encapsulated metabolite, sugar, amino acid, nucleoside, nucleotide, ribonucleoside, ribonucleotide or nutrient and the shell is silica. In some embodiments of the invention, a nano-powder of the desired metabolite, sugar, amino acid, nucleoside, nucleotide, ribonucleoside, ribonucleotide or nutrient is obtained by wet high energy ball milling grinding process or by dry high energy ball milling grinding process.

In some embodiments of the invention, the method described herein, further comprise a step of attaching or conjugating the nanoparticle or nanosphere or encapsulating shell to a T cell or NK cell targeting agent, which may be in some embodiments, an antibody, peptide, aptamer, heptamer, oligomer, targeting vector or nanobody.

In an embodiment of the invention, there is provided a method of enriching immune cells that are used in cellular immunotherapy, such as, without limitation, T cells, NK cells, TILs (Tumor Infiltrating Lymphocytes), CAR T cells, CAR or Natural killer cells (NK) cells or combination thereof with a required metabolite, sugar, amino acid, nucleoside, nucleotide, ribonucleoside, ribonucleotide or nutrient or a combination thereof. In some embodiments of the invention, there is provided a method of enriching modified and/or unmodified immune cells with a required metabolite, sugar, amino acid, nucleoside, nucleotide, ribonucleoside, ribonucleotide or nutrient or a combination thereof and/or a method of delivering into modified and/or unmodified immune cells a required metabolite, sugar, amino acid, nucleoside, nucleotide, ribonucleoside, ribonucleotide or nutrient or a combination thereof comprising the steps of contacting the modified and/or unmodified immune cells with a nanoparticle or nanosphere or a shell encapsulating with or attached to the required metabolite, sugar, amino acid, nucleoside, nucleotide, ribonucleoside, ribonucleotide or nutrient or the combination thereof, thereby enriching the modified or unmodified immune cells with a required metabolite, sugar, amino acid, nucleoside, nucleotide, ribonucleoside, ribonucleotide or nutrient or a combination thereof or delivering into the modified or unmodified immune cells a required metabolite, sugar, amino acid, nucleoside, nucleotide, ribonucleoside, ribonucleotide or nutrient or a combination thereof.

The nanoparticle, nanosphere or encapsulating shell is in some embodiments used to enhance the function, activation, life span and the like of the modified and/or unmodified immune cells for cancer immunotherapy treatment.

In some embodiments of the invention, the nanoparticle, nanosphere or encapsulating shell is contacted with the modified and/or unmodified immune cells either during the ex-vivo stage of adoptive modified and/or unmodified immune cells transfer therapy or targeted to modified and/or unmodified internal immune cells by systemic administration.

In some embodiments of the invention, the enriched immune cells are used for T cell or NK cell or CAR T or CAR NK or TIL adoptive cell transfer therapy.

In some embodiments of the invention, the nanoparticle, nanosphere or encapsulating shell gradually release the encapsulated required metabolite, sugar, amino acid, nucleoside, nucleotide, ribonucleoside, ribonucleotide or nutrient or the combination thereof into the immune cells. In some embodiments of the invention, a depot of the required metabolite, sugar, amino acid, nucleoside, nucleotide, ribonucleoside, ribonucleotide or a nutrient is created inside the modified and/or unmodified immune cells. In some embodiments of the invention, the nanoparticle, nanosphere or encapsulating shell are coated with or attached to modified and/or unmodified immune cells such as a T cell and/or NK cell targeting agent.

In some embodiments of the invention, the nanoparticle, nanosphere or shell encapsulating the metabolite, sugar, amino acid, nucleoside, nucleotide, ribonucleoside, ribonucleotide or nutrient are made of silica. The silica is in some embodiments, non-porous, porous, semi-porous, macro-porous or meso-porous and in some embodiments, may be polymerized using a sol-gel polymerization process.

In some embodiments of the invention, there is provided a method of delivering into modified and/or unmodified immune cells a required metabolite, sugar, amino acid, nucleoside, nucleotide, ribonucleoside, ribonucleotide or nutrient or a combination thereof, comprising the steps of contacting the modified and/or unmodified immune cells or a combination thereof with a nanoparticle or nanosphere or an encapsulating shell with or attached to the required metabolite, sugar, amino acid, nucleoside, nucleotide, ribonucleoside, ribonucleotide or nutrient or the combination thereof, thereby enriching the modified and/or unmodified immune cells with a required metabolite, sugar, amino acid, nucleoside, nucleotide, ribonucleoside, ribonucleotide or nutrient or a combination thereof or delivering into the modified and/or unmodified immune cells a required metabolite, sugar, amino acid, nucleoside, nucleotide, ribonucleoside, ribonucleotide or nutrient or a combination thereof.

In some embodiments of the invention, the nanoparticle, nanosphere or encapsulating shell are used to enhancing cells that are used in cellular cancer immunotherapy, such as, without limitation T cells, NK cells, TILs, CAR T cells, CAR NK cells or a combination thereof.

In some embodiments of the invention, the nanoparticle, nanosphere or shell is contacted with modified and/or unmodified immune cells that are used in cellular cancer immunotherapy, such as, without limitation T cells, NK cells, TILs, CAR T cells, CAR NK cells or a combination thereof either during the ex-vivo stage of adoptive modified immune cells, such as, T cell, NK cell, TILs, CART cell, CAR NK cell or combination thereof transfer therapy or targeted to modified and/or unmodified immune cells, such as, T cells, NK cells, TILs, CAR T cells, CAR NK cells or a combination thereof by systemic administration (e.g. intravenous administration).

In some embodiments of the invention, the enriched immune cells such as, T cells or NK cells that are contacted with the enriched nanoparticles, nanospheres or shell are used for CAR T or CAR NK or TIL adoptive cell transfer therapy. In some embodiments of the invention, the enriched nanoparticles, nanospheres or shell are injected systemically or locally and are targeted to immune cells, such as, without limitation T cell or NK cells or TILs.

In some embodiments of the invention, the enriched cells that are used in cellular cancer immunotherapy, are administered in combination with other treatment of cancer, such as, non-cellular immunotherapy approach like immune checkpoint inhibitors, cancer vaccines, conjugated antibodies, bi-specific T cell engagers, bi-specific NK cell engagers, oncolytic viruses, ‘eat me’ signals, ‘find me’ signals or others, or non-immunotherapy anti-cancer treatments, including chemotherapy, biological therapies like, for example, tyrosine kinase inhibitors, anti-angiogenic therapy, hormonal therapy, radiotherapy or surgery.

In some embodiments of the invention, the nanoparticle, nanosphere or shell gradually release the encapsulated required metabolite, sugar, amino acid, nucleoside, nucleotide, ribonucleoside, ribonucleotide or nutrient or the combination thereof in a controlled release manner with a release kinetics ranging from hours to days and weeks.

In some embodiments of the invention, a depot of the required metabolite, sugar, amino acid, nucleoside, nucleotide, ribonucleoside, ribonucleotide or nutrient or the combination thereof is created inside the enriched cells that are used in cellular cancer immunotherapy.

In some embodiments of the invention, the term “required metabolite, sugar, amino acid, nucleoside, nucleotide, ribonucleoside, ribonucleotide or a nutrient” means is an intermediate or end product of metabolism comprises, without limitation one or more of the following: glucose, fructose, galactose, glycerol, glutamine, glutamate, arginine, citrulline, serine, cysteine, tryptophan, alanine, histidine, lysine, aspartic acid, glutamic acid, threonine, asparagine, selenocysteine, glycine, proline, valine, leucine, isoleucine, methionine, phenylalanine, tyrosine, NAD, NADH (nicotinamide adenine dinucleotide), FAD, FADH2 (flavin adenine dinucleotide), glycolysis intermediates: glucose-6-phosphate, fructose-6-phosphate, fructose 1,6-biphosphate, dihydroxyacetone phosphate, glycerol 3-phosphate, 1,3-bisphosphoglycerate, 3-phosphoglycerate, 2-phosphoglycerate, phosphoenolpyruvate, pyruvate, citric acid cycle (Krebs cycle) intermediates: acetyl-CoA, citrate, cis-aconitate, isocitrate, alpha-ketoglutarate, succinyl-CoA, succinate, fumarate, malate, oxaloacetate, one-carbon metabolic pathway intermediates, folate cycle intermediates, methionine cycle intermediates, trans-sulfuration pathway intermediates, urea cycle intermediates, nucleotide synthesis pathway intermediates, nucleotide or nucleoside triphosphate, diphosphate or monophosphate, ribonucleotide or ribonucleoside triphosphate, diphosphate or monophosphate, ATP (adenosine triphosphate), GTP, CTP, UTP, TTP, dGTP, dCTP, dUTP or dTTP and a like.

In some embodiments of the invention, the “enriched nanoparticles, nanospheres or shell” refer to the nanoparticles, nanospheres or shell of the invention, i.e. nanoparticles, nanospheres or encapsulating shell containing one or more of a required metabolite, sugar, amino acid, nucleoside, nucleotide, ribonucleoside, ribonucleotide or a nutrient.

In some embodiments of the invention, the enriched nanoparticle, nanosphere or encapsulating shell is coated with or attached to modified and/or unmodified immune cell targeting agent.

In some embodiments of the invention, the targeting agent is an antibody, peptide, aptamer, heptamer, oligomer, targeting vector or nanobody.

In some embodiments of the invention, when the immune cells are T cells the antibody is an anti-CD3 antibody (e.g. OKT3, UCHT1, IP26, SK7, HIT3a or other clones) and/or an anti-CD4 antibody and/or an anti-CD8 antibody and/or an anti-PD1 antibody and/or an anti-CTLA4 antibody and the like or a combination thereof. The antibodies can be either monoclonal or polyclonal. If the immune cells are NK cells, the antibody is an anti-KIR antibody and/or an anti-CD16 antibody and/or an anti-CD94 antibody and/or an anti-CD161 antibody and/or an anti-CD56 antibody and the like or a combination thereof.

In some embodiments of the invention, the nanoparticle, nanosphere or shell encapsulating the metabolite, sugar, amino acid, nucleoside, nucleotide, ribonucleoside, ribonucleotide or a nutrient are made of silica or organically modified silica.

In some embodiments of the invention, the nanoparticle, nanosphere or shell encapsulating the metabolite, sugar, amino acid, nucleoside, nucleotide, ribonucleoside, ribonucleotide or a nutrient are made from tetraethyl orthosilicate (TEOS) or tetramethyl orthosilicate (TMOS) or sodium silicate or potassium silicate or alkoxysilane or silane or organically modified alkoxysilane or organically modified silane or a combination thereof.

In some embodiments of the invention, the nanosphere encapsulates the metabolite, sugar, amino acid, nucleoside, nucleotide, ribonucleoside, ribonucleotide or nutrient in its hollow core while the shell is made of silica. In some embodiments of the invention, the nanosphere encapsulates the metabolite, sugar, amino acid, nucleoside, nucleotide, ribonucleoside, ribonucleotide or nutrient in its hollow core, wherein the shell is made from tetraethyl orthosilicate (TEOS), tetramethyl orthosilicate (TMOS), sodium silicate, potassium silicate, alkoxysilane, silane, organically modified alkoxysilane, organically modified silane or a combination thereof.

In some embodiments of the invention, the nanoparticle is a core-shell structure made of silica shell grown on a substrate core comprising the required metabolite, sugar, amino acid, nucleoside, nucleotide, ribonucleoside, ribonucleotide, nutrient or a combination thereof. In some embodiments of the invention, the nanoparticle is a core-shell structure in which the shell is made from tetraethyl orthosilicate (TEOS) or tetramethyl orthosilicate (TMOS) or sodium silicate or potassium silicate or alkoxysilane or silane or organically modified alkoxysilane or organically modified silane or a combination thereof grown on a substrate core comprising the required metabolite, sugar, amino acid, nucleoside, nucleotide, ribonucleoside, ribonucleotide, nutrient or a combination thereof.

In some embodiments of the invention, there is provided a nanoparticle, nanosphere or a shell coated by or attached to by an immune cell targeting agent, wherein the nanoparticle or the nanosphere are loaded with a required metabolite, sugar, amino acid, nucleoside, nucleotide, ribonucleoside, ribonucleotide or nutrient or a combination thereof.

In some embodiments of the invention, the targeting agent is wherein the targeting agent is an antibody, peptide, aptamer, heptamer, oligomer, targeting vector or nanobody.

In some embodiments of the invention, when the immune cells are T cells the antibody is an anti-CD3 antibody (e.g. OKT3, UCHT1, IP26, SK7, HIT3a or other clones) and/or an anti-CD4 antibody and/or an anti-CD8 antibody and/or an anti-PD1 antibody and/or an anti-CTLA4 antibody and the like or a combination thereof. The antibodies can be either monoclonal or polyclonal. If the immune cells are NK cells the antibody is an anti-KIR antibody and/or an anti-CD16 antibody and/or an anti-CD94 antibody and/or an anti-CD161 antibody and/or an anti-CD56 antibody and the like or a combination thereof.

In some embodiments of the invention, nanoparticle or nanosphere are made of silica shell grown on the required metabolite, sugar, amino acid, nucleoside, nucleotide, ribonucleoside, ribonucleotide or the nutrient powder or nano-powder.

In some embodiments of the invention, there is provided a nanoparticle, nanosphere or encapsulating shell loaded with a required metabolite, sugar, amino acid, nucleoside, nucleotide, ribonucleoside, ribonucleotide or nutrient or a combination thereof. The nanoparticle, nanosphere or shell is in some embodiments coated by or attached to an immune cell targeting agent, wherein the targeting agent is an antibody, peptide, aptamer, heptamer, oligomer, targeting vector or nanobody.

In some embodiments of the invention, the nanoparticle or nanosphere are made of silica shell grown on a required metabolite, sugar, amino acid, nucleoside, nucleotide, ribonucleoside, ribonucleotide or a nutrient or combination thereof, or the nutrient powder or nano-powder.

In some embodiments of the invention, the nanoparticle or nanosphere releases the required metabolite, sugar, amino acid, nucleoside, nucleotide, ribonucleoside, ribonucleotide or a nutrient or the combination thereof in a controlled manner with a time frame of 0 minutes-24 hours or 2-10 or more days. In some embodiments of the invention, the nanoparticle, nanosphere or shell release the metabolite, sugar, amino acid, nucleoside, nucleotide, ribonucleoside, ribonucleotide, nutrient or the combination thereof in a controlled manner with a time frame of 0 minutes-24 hours and/or 1-20 or more days. The release may initiate once the nanoparticle, nanosphere or encapsulating shell is in contact with a solution including water, cell media and the like.

In some embodiments of the invention, the nanoparticle or the nanosphere is a non-porous silica nanoparticle, porous silica nanoparticle, semi-porous silica nanoparticle, core-shell nanoparticle, silica hollow sphere or coated.

In some embodiments of the invention, the silica is polymerized using a sol-gel process.

In some embodiments of the invention, tetraethyl orthosilicate (TEOS) or tetramethyl orthosilicate (TMOS) or sodium silicate or potassium silicate or alkoxysilane or silane or organically modified alkoxysilane or organically modified silane or a combination thereof are used as precursors (as building blocks) for the polymerization of the silica.

In some embodiments of the invention, an acid (e.g. HCl, HNO₃, H₂SO₄ or other strong or weak acids) or a base (e.g. NH₄OH, NaOH, KOH or other strong or weak bases) is used for catalyzing the silica formation.

In some embodiments of the invention, water is added to enable silica precursor hydrolysis followed by silica condensation.

In some embodiments of the invention, thermal heating is applied to adjust the silica properties.

In some embodiments of the invention, there is provided a method of manufacturing the nanoparticle, nanosphere or shell of the invention, comprising the steps of:

mixing NH₄OH and an alcohol for alkaline catalysis or mixing an acid (e.g. HCl, HNO₃, H₂SO₄) and an alcohol for acid catalysis;
optionally adding water;
adding tetraethyl orthosilicate (TEOS) or tetramethyl orthosilicate (TMOS) or sodium silicate or potassium silicate or alkoxysilane or silane or organically modified alkoxysilane or organically modified silane;
suspending the obtained nanoparticle or nanosphere in a water or glycerol or alcohol solution possibly with some water and adding the required metabolite, sugar, amino acid, nucleoside, nucleotide, ribonucleoside, ribonucleotide, nutrient or the combination thereof;
heating; and
lyophilizing.

In some embodiments of the invention, the alcohol is methanol, ethanol, 1-propanol, 2-propanol, butanol (linear or branched), pentanol (linear or branched), hexanol (linear or branched), a longer chain alcohol or a combination thereof.

In some embodiments of the invention, there is provided a method of manufacturing the nanoparticle, nanosphere or shell of the invention, comprising the steps of: grinding desired metabolite to the nano-scale using an alcohol for wet grinding or grinding desired metabolite to the nano-scale using dry grinding and suspending the metabolite nano-powder in an alcohol;

optionally adding a base (e.g. NH₄OH) for alkaline catalysis or adding an acid (e.g. HCl, HNO₃, H₂SO₄) for acid catalysis;
optionally adding water;
adding tetraethyl orthosilicate (TEOS) or tetramethyl orthosilicate (TMOS) or sodium silicate or potassium silicate or alkoxysilane or silane or organically modified alkoxysilane or organically modified silane;
optionally heating;
separating the nanoparticles; and
lyophilizing.

In some embodiments of the invention, the alcohol is methanol, ethanol, 1-propanol, 2-propanol, butanol (linear or branched), pentanol (linear or branched), hexanol (linear or branched), a longer chain alcohol or a combination thereof.

In some embodiments of the invention, the separation is by centrifugation.

In some embodiments of the invention, there is provided a method for manufacturing a nanoparticle, nanosphere or shell, which is hollow sphere comprising the steps of: mixing polyvinylpyrrolidone, styrene and water;

heating;
adding potassium persulfate or AIBA (2,2′-Azobis(2-methylpropionamidine) dihydrochloride) as initiator;
cooling;
adding ammonium hydroxide (for alkaline catalysis) or an acid (for acid catalysis), alcohol and TEOS or TMOS or sodium silicate or potassium silicate or alkoxysilane or silane or organically modified alkoxysilane or organically modified silane;
etching of the polystyrene core by calcination (above 350° C.) or by adding a solvent to dissolve the polystyrene core (e.g. toluene);
washing with an alcohol;
subjecting the formed hollow sphere to a metabolite, sugar, amino acid, nucleoside, nucleotide, ribonucleoside, ribonucleotide, nutrient or combination thereof loading solution;
heating;
drying; and optionally,
lyophilizing.

In some embodiments of the invention, if subjecting the formed hollow sphere to more than one metabolite, sugar, amino acid, nucleoside, nucleotide, ribonucleoside, ribonucleotide, nutrient or combination thereof, the process is conducted for each metabolite, sugar, amino acid, nucleoside, nucleotide, ribonucleoside, ribonucleotide, nutrient or combination thereof separately or jointly.

In some embodiments of the invention, once a nanoparticle, nanosphere or shell is formed, according to any of the methods, there is a further step of chemical derivatizing the resulted nanoparticle, nanosphere or shell.

Further, in some embodiments and there is a step of attaching or conjugating the nanoparticle, nanosphere or shell to an immune cell targeting agent.

In some embodiments of the invention, there is provided a method of manufacturing nanoparticle, nanosphere or encapsulating shell, having a core, wherein the core comprising a required metabolite, sugar, amino acid, nucleoside, nucleotide, ribonucleoside, ribonucleotide or a nutrient or the combination thereof that is encapsulated by a silica shell, comprising the steps of:

grinding the required metabolite, sugar, amino acid, nucleoside, nucleotide, ribonucleoside, ribonucleotide or a nutrient or the combination thereof to a nano-scale optionally in the presence of an alcohol for wet grinding to form a metabolite nano-powder;
suspending the metabolite nano-powder in an alcohol;
optionally adding a base for an alkaline catalysis or an acid for acid catalysis;
optionally adding water;
adding tetraethyl orthosilicate (TEOS), tetramethyl orthosilicate (TMOS), an alkoxysilane, sodium silicate, potassium silicate, silane, organically modified alkoxysilane or organically modified silane;
optionally heating;
washing the nanoparticles with alcohol;
separating the nanoparticles; and
lyophilizing.

In some embodiments of the invention, the grinding is by wet high energy ball milling grinding process or by dry high energy ball milling grinding process.

In some embodiments of the invention, there is provided a method for manufacturing nanoparticle, nanosphere or encapsulating shell encapsulating a required metabolite, sugar, amino acid, nucleoside, nucleotide, ribonucleoside, ribonucleotide or a nutrient or the combination thereof in an interior hollow space therein comprising the steps of:
mixing polyvinylpyrrolidone, styrene and water;
heating;
adding potassium persulfate or AMA (2,2′-Azobis(2-methylpropionamidine) dihydrochloride) as initiator;
cooling;
adding ammonium hydroxide for alkaline catalysis or an acid for acid catalysis;
adding an alcohol;
optionally adding water;
optionally adding a templating agent to control porosity;
adding tetraethyl orthosilicate (TEOS), tetramethyl orthosilicate (TMOS), analkoxysilane, sodium silicate, potassium silicate, silane, organically modified alkoxysilane or organically modified silane;
washing with an alcohol;
etching a polystyrene core by calcination at a temperature of above 350 C or by adding a solvent to dissolve the polystyrene core;
washing with an alcohol;
subjecting the formed hollow sphere to a loading solution comprising a required metabolite, sugar, amino acid, nucleoside, nucleotide, ribonucleoside, ribonucleotide, nutrient or combination thereof;
optionally heating;
drying; and
optionally lyophilizing.

In some embodiments of the invention, there is provided a method of manufacturing nanoparticle, nanosphere or encapsulating shell loaded with a required metabolite, sugar, amino acid, nucleoside, nucleotide, ribonucleoside, ribonucleotide or a nutrient or combination thereof, wherein the nanoparticle, nanospere or encapsulating shell is Stober-like comprising the steps of:

mixing NH₄OH and an alcohol for alkaline catalysis or mixing an acid and an alcohol for acid catalysis;
optionally adding water;
adding tetraethyl orthosilicate (TEOS) or tetramethyl orthosilicate (TMOS), analkoxysilane, sodium silicate, potassium silicate, silane, organically modified alkoxysilane or organically modified silane;
suspending the obtained nanoparticles in water, glycerol, alcohol, glycerol in combination with water or alcohol in combination with water together with the desired metabolite, sugar, amino acid, nucleoside, nucleotide, ribonucleoside, ribonucleotide, nutrient or the combination thereof;
heating;
washing with water and/or alcohol; and
lyophilizing.

In some embodiments of the invention, there is provided a method of manufacturing nanoparticle, nanosphere or encapsulating shell, wherein a required metabolite, sugar, amino acid, nucleoside, nucleotide, ribonucleoside, ribonucleotide or a nutrient or combination thereof is encapsulated in a silica shell, comprising of steps:

mixing an alcohol with NH₄OH for alkaline catalysis or adding an acid for acid catalysis; optionally adding water;
adding tetraethyl orthosilicate (TEOS), tetramethyl orthosilicate (TMOS), an alkoxysilane, sodium silicate, potassium silicate, silane, organically modified alkoxysilane or organically modified silane;
optionally heating;
washing the nanoparticles with alcohol;
separating the nanoparticles;
suspending the obtained nanoparticles in water or alcohol and water solution with a required metabolite, sugar, amino acid, nucleoside, nucleotide, ribonucleoside, ribonucleotide, nutrient or the combination thereof;
adding tetraethyl orthosilicate (TEOS), tetramethyl orthosilicate (TMOS), an alkoxysilane, sodium silicate, potassium silicate, silane, organically modified alkoxysilane or organically modified silane;
heating;
washing with water and/or alcohol; and
lyophilizing.

The alcohol is in some embodiments of the invention, methanol, ethanol, 1-propanol, 2-propanol, butanol (linear or branched), pentanol (linear or branched), hexanol (linear or branched), a longer chain alcohol (branched or not) or a combination thereof.

In some embodiments of the invention, the separation is by centrifugation.

In some embodiments of the invention, the methods for manufacturing the nanoparticle, nanosphere or encapsulating shell further comprising a step of chemical derivatization of the resulted nanoparticles, nanospheres or encapsulating shell.

In some embodiments of the invention, the methods for manufacturing the nanoparticle, nanosphere or encapsulating shell further comprising a step of attaching or conjugating the nanoparticle, nanosphere or encapsulating shell to immune cell targeting agent.

In some embodiments of the invention, the nanoparticle, nanosphere or encapsulating shell the nanoparticle, nanosphere or encapsulating shell is further PEGylated.

### EXAMPLES

### Metabolic Nutrient Encapsulation in Silica

### Example 1: Growing Silica Shell on Metabolite Nano-Powder

Arginine.HCl (1119-34-2) was milled using Emax High Energy Ball Mill (Retsch) to yield nano-powder: 10 gr of Arginine.HCl and 110 gr of 5 mm grinding balls and 20 ml isopropyl alcohol were added to a 50 ml zirconia grinding jar. Powder was grinded for one hour with a speed of 1000 rpm, followed by addition of 5 ml isopropyl alcohol and additional grinding for two hours with 110 gr of 0.5 mm grinding balls at a speed of 1800 rpm. One gr of Arginine.HCl nano-powder was dispersed in 20 ml EtOH absolute and 800 ul ammonium hydroxide 25% was added. Then, 100 ul TEOS was added every 30 minutes for a total of 10 additions (1 ml TEOS in total). Silica shell was allowed to form for 24 hours. Nanoparticles were precipitated and washed twice with ethanol and once with DDW and dried by lyophilization.

Arginine (CAS 74-79-3) was milled using Emax High Energy Ball Mill (Retsch) to yield nano-powder: 10 gr of Arginine and 110 gr of five mm grinding balls and 20 ml isopropyl alcohol were added to a 50 ml zirconia grinding jar. Powder was grinded for one hour with a speed of 1000 rpm, followed by addition of 5 ml isopropyl alcohol and additional grinding for two hours with 110 gr of 0.5 mm grinding balls at a speed of 1800 rpm. One gr of Arginine nano-powder was dispersed in 20 ml EtOH absolute and 800 ul ammonium hydroxide 25% was added. Then, 100 ul TEOS was added every 30 minutes for a total of 10 additions (one ml TEOS in total). Silica shell was allowed to form for 24 hours. Nanoparticles were precipitated and washed twice with ethanol and once with DDW and dried by lyophilization.

Glucose was milled using Emax High Energy Ball Mill (Retsch) to yield nano-powder: 10 gr of glucose and 110 gr of 5 mm grinding balls and 20 ml isopropyl alcohol were added to a 50 ml zirconia grinding jar. Powder was grinded for one hour with a speed of 1000 rpm, followed by addition of five ml isopropyl alcohol and additional grinding for two hours with 110 gr of 0.5 mm grinding balls at a speed of 2000 rpm. One gr of glucose nano-powder was dispersed in 20 ml EtOH absolute and 800 ul ammonium hydroxide 25% was added. Then, 100 ul TEOS was added every 30 minutes for a total of 10 additions (1 ml TEOS in total). Silica shell was allowed to form for 24 hours. Nanoparticles were precipitated and washed twice with ethanol and once with DDW and dried by lyophilization.

Glutamine was milled using Emax High Energy Ball Mill (Retsch) to yield nano-powder: 10 gr of glutamine and 110 gr of 5 mm grinding balls and 20 ml isopropyl alcohol were added to a 50 ml zirconia grinding jar. Powder was grinded for one hour with a speed of 1000 rpm, followed by addition of 5 ml isopropyl alcohol and additional grinding for two hours with 110 gr of 0.5 mm grinding balls at a speed of 1800 rpm. 1 gr of glutamine nano-powder was dispersed in 20 ml EtOH absolute and 800 ul ammonium hydroxide 25% was added. Then, 100 ul TEOS was added every 30 minutes for a total of 10 additions (one ml TEOS in total). Silica shell was allowed to form for 24 hours. Nanoparticles were precipitated and washed twice with ethanol and once with DDW and dried by lyophilization.

Serine was milled using Emax High Energy Ball Mill (Retsch) to yield nano-powder: 10 gr of serine and 110 gr of 5 mm grinding balls and 20 ml isopropyl alcohol were added to a 50 ml zirconia grinding jar. Powder was grinded for one hour with a speed of 1100 rpm, followed by addition of five ml isopropyl alcohol and additional grinding for two hours with 110 gr of 0.5 mm grinding balls at a speed of 1900 rpm. One gr of serine nano-powder was dispersed in 20 ml EtOH absolute and 800 ul ammonium hydroxide 25% was added. Then, 100 ul TEOS was added every 30 minutes for a total of 10 additions (one ml TEOS in total). Silica shell was allowed to form for 24 hours. Nanoparticles were precipitated and washed twice with ethanol and once with DDW and dried by lyophilization.

### Example 2: Metabolic Nutrient Loaded Silica Nanoparticles Using the Stober Process

Silica nanoparticles were synthesized using the Stober process: 13 ml NH₄OH 25% were added to 65 ml ethanol abs. followed by the addition of 2.6 ml TEOS. Nanoparticles were allowed to form and age overnight. The nanoparticles were then washed twice with ethanol abs. and twice with DDW. The nanoparticles then were suspended in a 40% glycerol solution (5 ml glycerol abs.+7.5 ml DDW) and 4 gr L-Arginine.HCl or 4 gr L-Arginine were added. Suspension was heated to 80° C. for 4 days. The resulted nanoparticles were washed twice with DDW and subjected to lyophilization.

### Example 3: Metabolic Nutrient Loaded Silica Core-Shell Nanoparticles

Under vigorous stirring, 2.9 ml NH₄OH 25% (Sigma) were added to 14.5 ml ethanol absolute. 580 ul TEOS (Tetraethyl orthosilicate, CAS 78-10-4, Sigma) were then added. Nanoparticles were allowed to form and age overnight. The following day, 2 gr L-Arginine.HCl (L-Arginine monohydrochloride, CAS 1119-34-2, Alfa) or 2 gr L-Arginine (CAS 74-79-3) were added. 50 ul TEOS were then added every 30 minutes for a total of 500 ul TEOS (10 additions). Suspension was allowed to age overnight. The following day, suspension was heated under reflux (−80° C., ethanol boiling point) for 48 hours. After cooling down, nanoparticles were precipitated using centrifugation (4000 g, 20 min, R.T.) and washed twice with 45 ml ethanol absolute followed by two washings with 45 ml DDW. Pellet was subject to lyophilization for 24 hours.

### Example 4: Metabolic Nutrient Loaded Hollow Silica Nano-Spheres

Hollow spheres were synthesized using hard-templating route: polystyrene cores were prepared as follows: 1.5 gr polyvinylpyrrolidone and 11 ml styrene were added to 90 ml DDW at room temperature under nitrogen flow. After 30 min, emulsion was gradually heated to 70° C. 10 ml DDW containing 0.1 gr potassium persulfate were then added. Reaction was held at 70° C. for 24 hours and then allowed to cool to room temperature. Silica shell was grown on polystyrene cores as follows: 5.5 ml polystyrene suspension and 1 ml ammonium hydroxide 25% were added to 120 ml ethanol abs. 200 ul TEOS (Tetraethyl orthosilicate) were added every 15 min to the reaction for a total of 30 additions (6 ml TEOS). Reaction was allowed to age overnight. The nanoparticles were then washed two times with ethanol absolute and subjected to calcination at 550 C for three hours to obtain hollow silica spheres. 45 mg hollow spheres were added to a 0.5 gr/ml glucose aqueous solution with stirring for seven days to enable glucose loading. 45 mg hollow spheres were added to a 0.2 gr/ml arginine aqueous solution with stirring for seven days to enable arginine loading. 45 mg hollow spheres were added to a 0.2 gr/ml glutamine aqueous solution with stirring for seven days to enable glutamine loading. 45 mg hollow spheres were added to a 0.2 gr/ml serine aqueous solution with stirring for 7 days to enable serine loading. After loading, nanoparticles were washed twice with DDW and once with ethanol abs. and dried under nitrogen flow at room temperature.

### Example 5: Nanoparticle Surface Derivatization

45 mg nanoparticles (arginine loaded or control) were suspended in nine ml 96% ethanol. 150 ul APTES ((3-Aminopropyl)triethoxysilane, CAS 919-30-2, Sigma) were added and stirred overnight at R.T. One wash with 45 ml ethanol absolute followed by a wash with 45 ml 70% ethanol.

### Example 6: Anti-CD3 Conjugation to Nanoparticles

Following derivatization, nanoparticles were suspended in one ml DDW containing 12 mg NHS (N-Hydroxysuccinimide, CAS 6066-82-6, Sigma). 71 ul EDC (1-Ethyl-3-(3-dimethylaminopropyl)carbodiimide, CAS 1892-57-5, Sigma) were added. Swirling for 15 minutes at R.T. Centrifugation and discarding supernatant. Re-suspension in 1 ml 10 mM Glycine buffer, pH 5.0. Addition of 2.5 ul anti-CD3 antibody (Ultra-LEAF™ Purified anti-human CD3 Antibody OKT3 clone, one ug/ul, Biolegend). Swirling for one hour at R.T. Centrifugation and discarding supernatant. Addition of one ml Ethanolamine 1M. Swirling for 10 minutes at R.T. Two washes with 45 ml phosphate buffer followed by re-suspension of nanoparticles in Complete Lymphocytes Medium (CLM).

### Example 7: Controlled Release Kinetics for Arginine and Glucose from Silica Nanoparticles Feeding Nanoparticles to T Cells

30 million human CD8+ T cells following REP (rapid expansion) were thawed and cultured in Complete Lymphocyte Medium (CLM) with 1,000 IL-2 units/ml three days prior to experiment. Anti-CD3 conjugated nanoparticles (no arginine control, arginine nanoparticles type 1, arginine nanoparticles type 2) were suspended each in 5 ml CLM. T-cells were cultured 200,000 cells per well in 96 well plate in 200 ul CLM (containing arginine). To each well 1, 5, 10, 20 or 50 ul of nanoparticle suspension in CLM were added. Cells were allowed to uptake nanoparticles overnight.

### Preparation of Arginine Depleted Medium Using Human Arginase 1

48 ul of human arginase 1 (25 ug, ProspecBio) were added to 8 ml of CLM and incubated overnight in 370 C incubator. Arginine depleted CLM was sampled twice and analyzed by LC-MS to confirm arginine depletion. Arginine signal was at the instrument's noise level.

### Coating Anti-CD3 Antibody on 96 Well Plate

96 well plate was coated with anti-CD3 (OKT3 as above, one ug/ml in PBS, 100 ul per well) for one hour at R.T. Then each well was washed once with 200 ul PBS.

### Arginine Starvation Suppresses T Cell Activation

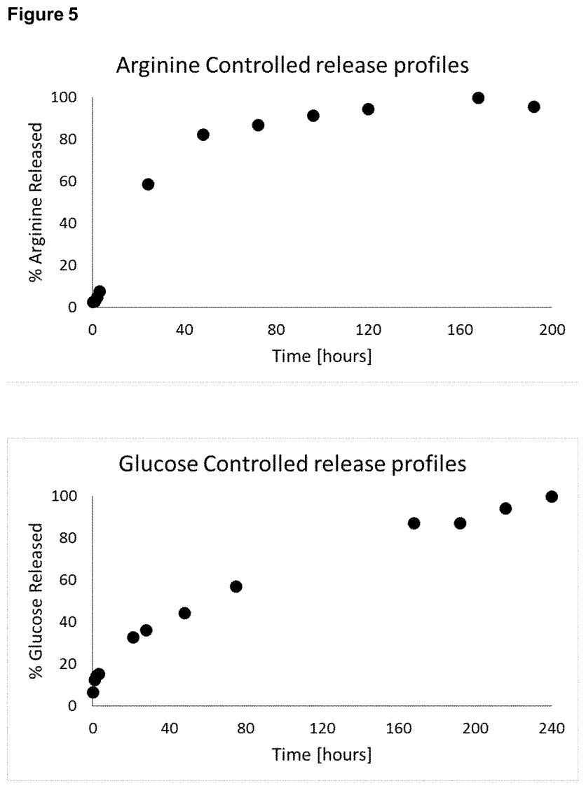

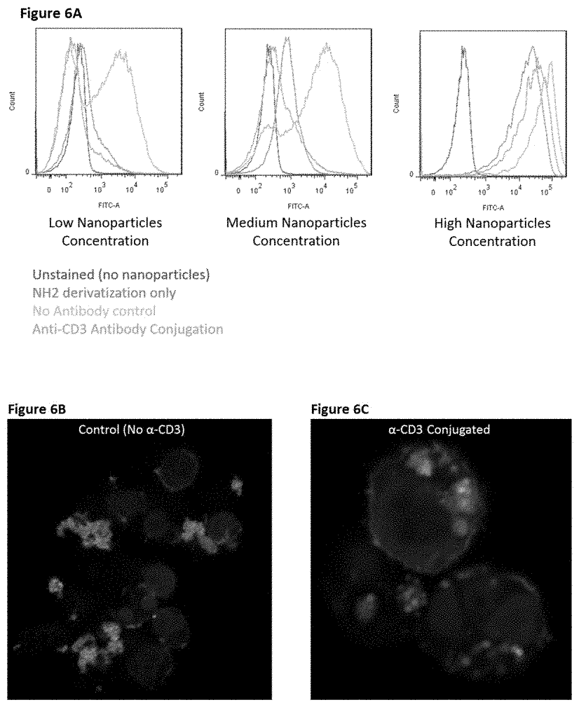

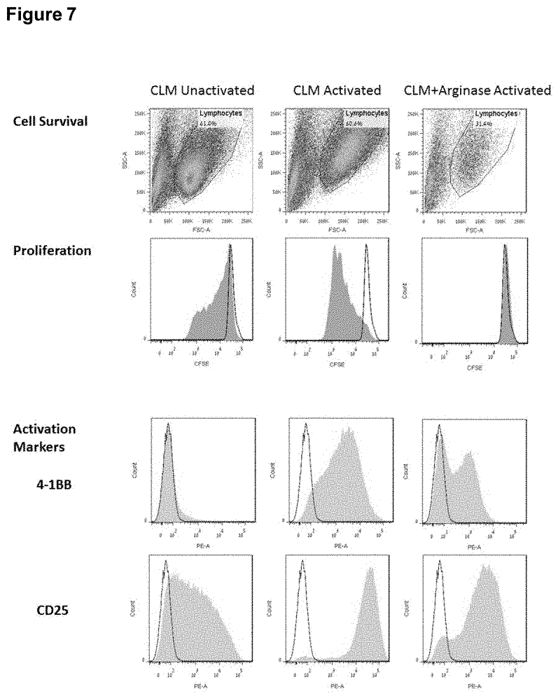

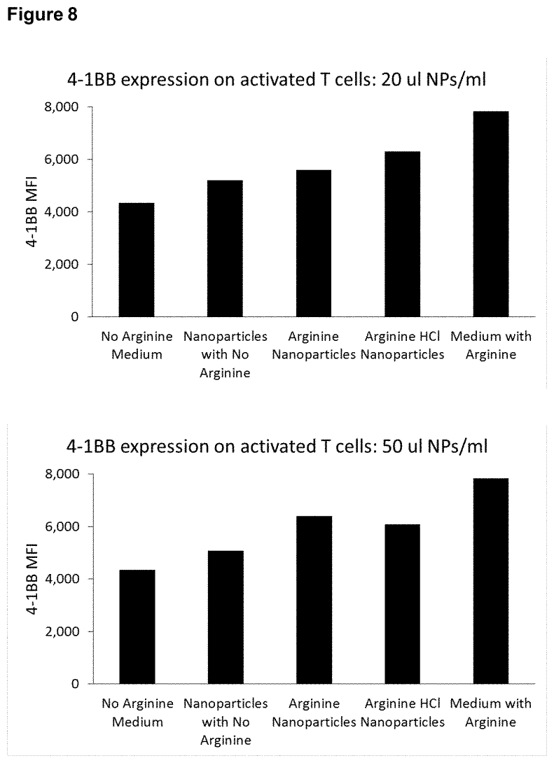

Using human arginase 1 enzyme (ProspecBio), Complete Lymphocyte Medium (CLM) was depleted of arginine (confirmed by LC-MS). Human CD8⁺ T-cells were activated using anti-CD3 (OKT3 MAb) and anti-CD28 stimulation in standard CLM (containing arginine) vs. arginine-depleted CLM (FIG. 5). Staining for T cell activation markers was done 24 hours following activation. T cell survival using was evaluated 48 hours following activation. Proliferation was assayed by CFSE staining before activation and analysis after 96 hours. The results show that arginine depletion causes an increase in T cell apoptosis, proliferation arrest and reduced expression of activation markers. This demonstrates the dependency of T cell function on arginine concentration, thereby enabling further experiments on nanoparticle enhancement of activity.

### Restoration of T Cell Functions Using Arginine Encapsulating Silica Nanoparticles

T cells fed with nanoparticles were transferred to sterile tubes and washed three times with three ml PBS. Then cells were suspended in 200 ul CLM medium with no arginine. Cells were returned to a 96 plate coated with anti-CD3 antibody and allowed to undergo activation for 24 hours. Cells were then extracellularly stained for 4-1BB activation marker: cells were transferred to FACS tubes and washed once with three ml FACS buffer. 15 min staining at R.T. with 2.5 ug PE conjugated anti-4-1BB (Biolegend) per tube. One wash with three ml FACS and one wash final wash with three ml PBS. Cells were suspended in 300 ul PBS and analyzed by LSR-II flow cytometer.

### Example 8: Feeding Nanoparticles to NK Cells

NK cells are isolated from human whole blood using negative selection kit for NK separation (EasySep™ Direct Human NK Cell Isolation Kit, StemCell Technologies) and cultured in CLM with 1,000 IL-2 units/ml. Anti-KIR conjugated nanoparticles (no arginine control, arginine nanoparticles type 1, arginine nanoparticles type 2) are suspended each in CLM. NK cells are cultured in 96 well plate in CLM (containing arginine). To each well nanoparticle suspension in CLM are added. Cells are allowed to uptake nanoparticles overnight. The tests are performed as described for the T-Cells with the required slight modifications if required.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1.-42. (canceled)

43. A method of enriching immune cells with a required substance, the method comprising the step of contacting the immune cells with a nanoparticle comprising the required substance, thereby enriching the immune cells with the required substance, wherein the required substance is a required metabolite, sugar, amino acid, nucleoside, nucleotide, ribonucleoside, ribonucleotide, nutrient, or any combination thereof.

44. The method of claim 43, wherein the substance is an amino acid and sugar, said amino acid is selected from: arginine, glutamine, serine, tryptophan, alanine, methionine, glycine or a combination thereof and wherein the sugar is glucose.

45. The method of claim 43, wherein the required substance comprises at least about 5% w/w of the weight of the nanoparticle.

46. The method of claim 43, wherein the enrichment of the immune cells with the required substance enhances one or more of: activity, viability, potency, life span or function of the immune cells.

47. The method of claim 43, wherein the nanoparticle is contacted with the immune cells during an ex-vivo stage or is targeted to internal immune cells by systemic administration.

48. The method of claim 43, wherein the immune cells are selected from the group consisting of: T cells, NK cells, CAR T cells, CAR NK cells, TIL cells, or any combination thereof.

49. The method of claim 43, wherein the nanoparticle is capable of gradually releasing the required substance into the immune cells.

50. The method of claim 43, wherein the nanoparticle comprises silica.

51. The method of claim 43, wherein the nanoparticle is coated with or attached to an immune cell targeting agent.

52. The method of claim 51, wherein the immune cell targeting agent is selected from the group consisting of an antibody, peptide, aptamer, heptamer, oligomer, targeting vector, and combinations thereof.

53. A nanoparticle for enriching immune cells with a required substance, the nanoparticle comprising a silica shell, wherein the required substance comprises a required metabolite, sugar, amino acid, nucleoside, nucleotide, ribonucleoside, ribonucleotide, nutrient or the combination thereof.

54. The nanoparticle of claim 53, wherein the required substance comprises glucose, arginine, glutamine, serine, tryptophan, alanine, methionine, glycine or a combination thereof.

55. The nanoparticle of claim 53, wherein the required substance comprises at least about 5% w/w of the weight of the nanoparticle.

56. The nanoparticle of claim 53, wherein the nanoparticle is coated by or attached to an immune cell targeting agent, wherein the immune cell targeting agent is an antibody, peptide, aptamer, heptamer, oligomer, targeting vector, nanobody, or any combination thereof.

57. The nanoparticle of claim 53, wherein the nanoparticle is capable of releasing the required substance within the immune cells in a controlled manner with a time frame of 0 minutes-24 hours or 2-10 days.

58. The nanoparticle of claim 53, wherein the nanoparticle is capable of delivering the required substance to the immune cells.

59. A composition comprising a plurality of the nanoparticles according to claim 53.

60. A nanoparticle for enriching immune cells with sugar and/or an amino acid, the nanoparticle comprising a shell capable of encapsulating the sugar and/or the amino acid, wherein the nanoparticle is coated by or attached to one or more immune cell targeting agents, wherein the one or more targeting agent is selected from an antibody, peptide, aptamer, heptamer, oligomer, targeting vector or nanobody.

61. The nanoparticle of claim 60, wherein the sugar is glucose, and/or the amino acid is one or more of: arginine, glutamine, serine, tryptophan, alanine, methionine, glycine or a combination thereof.

62. The nanoparticle of claim 60, wherein the nanoparticle is capable of releasing the sugar and/or the amino acid in a controlled manner with a time frame of 0 minutes-24 hours or 2-10 days.

63. The nanoparticle according to claim 60, wherein the nanoparticle shell comprises silica.
